(19) United States
(12) Patent Application Publication
(10) Pub. No.: US 2016/0206551 A1
(43) Pub. Date: Jul. 21, 2016

(54) METHODS FOR THE REPAIR AND REJUVENATION OF TISSUES USING PLATELET-RICH PLASMA COMPOSITIONS

(71) Applicant: William K. BOSS, JR., Essex Fells, NJ (US)

(72) Inventor: William K. BOSS, JR., Essex Fells, NJ (US)

(21) Appl. No.: 14/597,704

(22) Filed: Jan. 15, 2015

(51) Int. Cl. A61K 8/98; A61M 37/00; A61Q 19/08; A61Q 7/00; A61Q 19/00; A61Q 19/02

(52) U.S. Cl. CPC A61K 8/983; A61K 8/981; A61Q 19/00; A61Q 19/02; A61Q 19/08; A61Q 7/00; A61M 37/0015; A61K 2800/91; A61K 2800/805; A61K 2800/882; A61K 2800/884; A61M 2037/0046; A61M 2037/0061

(57) ABSTRACT

This application relates to cosmetic methods for improving skin conditions. The methods generally include the administration of platelet-rich plasma and cultured autologous cells to a subject after a micro-needling procedure has been performed. The platelet-rich plasma and autologous cells can be topically applied to the skin surface or injected into the dermis or subcutaneous tissue. The methods may be used to improve conditions such as acne scars, traumatic scars, hyperpigmentation, fine lines, wrinkles, stretch marks, and hair loss.

### FIELD

This application relates to methods for administering platelet-rich plasma (PRP) compositions to subjects. Specifically, the methods may involve topical application of the PRP composition to the skin surface after a micro-needling procedure has been performed, or injection of the PRP composition into subcutaneous or dermal tissue. The PRP composition will generally include cultured autologous cells, e.g., cultured autologous fibroblasts. Conditions such as acne scars, traumatic scars, hyperpigmentation, fine lines, wrinkles, stretch marks, and hair loss may be treated using the compositions.

### BACKGROUND

The injection of synthetic material into the body, and particularly into the face, to effect an aesthetic result dates to the close of the nineteenth century. For example, the injection of paraffin to correct facial contour defects enjoyed a brief period of acceptance in the years prior to World War I. However, complications and the unsatisfactory nature of the long-term results caused the practice to be abandoned. Injectable silicone became available in the early 1960s, but complications such as local and systemic reactions to the silicone, migration of the injectate, and local tissue break down, limited its use.

The poor results obtained by the injection of non-biological materials prompted attempts to use foreign proteins, e.g., bovine atelocollagen, as injectable filler materials to correct skin defects and/or to cosmetically improve the appearance of skin. However, the use of atelocollagen proved to be less than satisfactory since absorption of the material occurred without replacement by host material, and patients often developed immune reactions and resistance to the atelocollagen with repeated injections. Due to the limitations of bovine atelocollagen, human collagen products derived from placenta or autologous tissue, and other autologous cellular compositions have been developed, but their use as injectates have also provided unsatisfactory results.

Over the past 15 to 20 years, another procedure used on damaged or aging skin to improve its appearance is micro-needling. The micro-needle device creates tiny wounds in the skin that stimulate collagen production by the natural healing process of the body. However, the amount of collagen produced by micro-needling is generally not as much as seen with other skin resurfacing techniques. Clinicians have also attempted to apply various types of cells on the skin after micro-needling. However, because the cells were not successfully absorbed, results were suboptimal.

Accordingly, it would be desirable to have alternative methods for treating or cosmetically improving skin conditions.

### SUMMARY

Described herein are methods for cosmetically improving skin conditions that include the administration of a platelet-rich plasma composition, an autologous cell composition, or combinations thereof, to the skin after a micro-needling procedure has been performed. The micro-needling creates holes in the skin surface, which improve absorption of the platelet-rich plasma or autologous cell composition beneath the skin surface, to thereby provide enhanced tissue regeneration. Given that the platelet-rich plasma is made from the subject's own blood, there is no risk of the subject developing an immune reaction to the composition. Another benefit of platelet-rich plasma is that is has the capability of building tissue over time instead of degenerating, as experienced with many of the compositions mentioned above. The methods and compositions described herein may also be useful in improving conditions other than skin conditions.

In general, the methods for cosmetically improving skin conditions comprise: 1) obtaining a blood sample from a subject affected by a skin condition; 2) preparing a platelet-rich plasma composition from the blood sample; 3) preparing an autologous cell composition; and 4) performing a micro-needling procedure over an area of skin affected by the skin condition followed by topical application of the platelet-rich composition and the autologous cell composition to the affected area. The methods may be useful in the treatment of skin conditions such as acne scars, traumatic scars, hyperpigmentation, fine lines, wrinkles, stretch marks, and hair loss.

As further described below, the platelet-rich plasma composition can serve as a carrier for autologous cell compositions. In this instance, suspensions of various autologous cell types can be created and combined with the platelet-rich plasma prior to topical application on the skin. An extracellular matrix material that provides a scaffold for the autologous cells can further be added to the platelet-rich plasma and autologous cell composition, or separately applied to the skin, or separately injected into the dermis or subcutaneous tissue. Various cellular growth factors can also be added to the PRP or autologous cell compositions, or applied separately in a composition to the skin surface, or separately injected into the dermis or subcutaneous tissue.

When not used as a carrier, the platelet-rich plasma composition and autologous cell composition can be layered in any desired order. For example, the platelet-rich plasma could be applied first followed by the autologous cell composition or vice versa. The layering of compositions can also be repeated as desired. A tissue sealant may be applied over any layer to help retain the topically applied PRP and/or autologous cell composition within the skin. In other instances, the platelet-rich plasma, autologous cell compositions, and/or extracellular matrix material are combined and injected into or beneath the skin, e.g., into the epidermis, dermis, or subcutaneous tissue to improve a skin condition. Other exemplary compositions that can be topically applied to the skin surface alone or with autologous cells and/or PRP may comprise cellular growth factors, extracellular matrix material such as human amnion extracellular matrix or porcine urinary bladder extracellular matrix, conditioned media, or combinations thereof. Again, the compositions can be layered in any desired order. These same compositions can also be injected into the dermis or subcutaneous tissue.

### DETAILED DESCRIPTION

Described herein are various methods for administering platelet-rich plasma (PRP) compositions to subjects for the rejuvenation of tissues, e.g., tissues of the skin. The methods will usually include forming micro-perforations in the skin surface to enhance absorption of the PRP compositions therein. Autologous cells can be added to the PRP compositions and then administered to the subject, or the cells can be administered as a separate composition, e.g., in a layer applied either before or after application of the PRP composition. The layering of the compositions may improve their absorption into the skin after micro-needling. Skin appearance may also be improved with the layered application of multiple cellular compositions or application of compositions comprising a plurality of cell types, in addition to PRP application. For example, skin laxity, poor skin tone, and dull complexion may be improved using the methods and compositions described herein.

### Micro-Needling

The micro-perforations will generally be formed by micro-needling devices. Any suitable micro-needling device may be used. For example, devices where the needles are disposed on a rotatable drum and manually rolled over the skin (dermarollers), radiofrequency micro-needling devices, or automated micro-needling devices may be used. Exemplary automated micro-needling devices include without limitation, the Dermapen® micro-needling device (Dermapen, Salt Lake City, Utah) and the Micropen™ micro-needling device (Eclipse Aesthetics, LLC, Dallas, Tex.). In some variations of the method, it may be beneficial to employ an automated micro-needling device. Automated micro-needling devices are capable of providing consistent needle depth penetration and are convenient to use over narrow areas of skin around the nose, eyes, or mouth. Prior to micro-needling with an automated device, a lubricant is typically applied to the skin surface. Exemplary lubricants include without limitation, compositions comprising one or more of the following: 1) hyaluronic acid; 2) adipose cells; 3) adipose derived mesenchymal cells; 4) fibroblasts; 5) PRP; and 6) conditioned media. In one variation, the lubricant may comprise PRP. In other variations, the lubricant may include adipose cells, adipose derived mesenchymal cells, or combinations thereof. In yet further variations, the lubricant may include conditioned media and also one or more of the following: 1) hyaluronic acid; 2) fibroblasts; 3) and stem cells. The application of a lubricant may help to evenly distribute the needle openings in the skin.

The automated micro-needling devices will generally include a sterile, disposable portion and a reusable handle. A plurality of needles is typically fixed on the disposable needle portion, which is removably secured to the reusable handle. The needle size may vary, but is typically 32-gauge. The devices may also be configured to include between six to 24 needles. In one variation, the micro-needling device comprises twelve 32-gauge needles. In other variations, the micro-needling device includes a needle depth adjustment mechanism that allows adjustment of needle depth between, e.g., about 0.5 mm to 2.75 mm. Needle depth may be tailored to suit the thickness of the skin in the affected area. For example, when the micro-needling procedure is performed on thinner skin such as fairer skin or skin around the eyes, the needle depth may be set to 0.5 mm. When the micro-needling procedure is performed on the thicker skin of the forehead, the needle depth may be set to between 2.5 mm to 2.75 mm. Other treatments or techniques for skin resurfacing and/or heating the skin, e.g., laser, radiofrequency micro-needling, or ultrasound, may be employed with the methods and compositions described herein.

### Platelet-Rich Plasma Compositions

As previously stated, the methods described herein include the administration of a PRP composition to a subject after a micro-needling procedure has been performed. PRP is an autologous preparation of platelets in concentrated plasma. PRP contains a high concentration of various growth factors, including platelet-derived growth factor (PDGF), transforming growth factor (TGF), vascular endothelial growth factor (VEGF), and insulin-like growth factor (IGF-1), which are secreted from the alpha-granules of platelets when activated. These factors are known to regulate numerous biological processes, including wound healing, and promote cell migration, attachment, proliferation and differentiation, as well as promote extracellular matrix formation.

The PRP composition can be prepared by any suitable technique known in the field. In brief, whole blood is initially drawn from a subject. The blood is centrifuged to separate the plasma from packed red blood cells and then further centrifuged to separate PRP from platelet-poor plasma. The PRP can then be collected and administered to the subject as described herein.

Exemplary devices for preparing PRP include the GPS® II Platelet Concentrate Separation Kit and the Plasmax™ Plus Plasma Concentrator accessory (Biomet Biologics, Inc., Warsaw, Ind.), and the Vortech™ Concentration System (Biomet Biologics, Inc., Warsaw, Ind.). The Selphyl® System (Aesthetic Factors LLC, Bethlehem, Pa.) and Eclipse PRP™ System (Eclipse Aesthetics, Dallas, Tex.) may be useful for office preparation of PRP. The concentration of platelets within the PRP may vary. For example, in some variations, the platelet concentration in the PRP can range from about 3-fold to about 10-fold greater than the platelet concentration in whole blood.

### Autologous Cell Compositions

Autologous cell compositions can be prepared and added to a carrier PRP composition, or separately applied before or after application of the PRP. The autologous cell compositions may comprise any suitable cell type, including but not limited to, adipose cells, adipose derived mesenchymal cells, chondrocytes, fibroblasts, keratinocytes, stem cells, and combinations thereof. It may be beneficial to include stem cells such as adipose stem cells, cells derived from bone marrow aspirate, or mesenchymal stem cells (e.g., adipose derived mesenchymal cells) in the compositions. In practice, any undifferentiated mesenchymal cell that can be expanded in culture can be used. For example, an autologous dermal fibroblast composition can be made by expanding the cells in culture and then adding them to a PRP composition for combined application, or expanded in culture and applied before or after the PRP composition.

To make an autologous dermal fibroblast composition, a dermal fibroblast culture is initiated from a 2×5 mm full thickness biopsy specimen of the skin. Before the initiation of the culture, the biopsy is washed repeatedly with antibiotic and antifungal agents. Thereafter, the epidermis and the subcutaneous adipocyte-containing tissue is removed, so that the resultant culture is substantially free of non-fibroblast cells, and the specimen of dermis is finely divided with scalpel or scissors. The pieces of the specimen are individually placed with a forceps onto the dry surface of a tissue culture flask and allowed to attach for between 5 and 10 minutes before a small amount of medium is slowly added, taking care not to displace the attached tissue fragments. After 24 hours of incubation, the flask is fed with additional medium. When a T-25 flask is used to start the culture the initial amount of medium is 1.5-2.0 ml. The establishment of a cell line from the biopsy specimen ordinarily takes between two to three weeks, at which time the cells can be removed from the initial culture vessel for expansion.

During the early stages of the culture it is desired that the tissue fragments remain attached to the culture vessel bottom. Any fragments that detach should be reimplanted into new vessels. The fibroblasts can be stimulated to grow by a brief exposure of the tissue culture to EDTA-trypsin, according to techniques well known to those skilled in the art. The exposure to trypsin is too brief to release the fibroblasts from their attachment to the culture vessel wall. Immediately after the cultures have become established and are approaching confluence, samples of the fibroblasts can be removed for frozen storage. The frozen storage of early rather than late passage fibroblasts is preferred because the number of passages in cell culture of normal human fibroblasts is limited.

The fibroblasts can be frozen in any freezing medium suitable for preserving fibroblasts. A medium consisting of 70% growth medium, 20% (v/v) fetal bovine serum and 10% (v/v) dimethylsulfoxide (DMSO) can be used with good effect. Thawed cells can be used to initiate secondary cultures to obtain suspensions for use in the same subject without the inconvenience of obtaining a second specimen.

Any tissue culture technique that is suitable for the propagation of dermal fibroblasts from biopsy specimens may be used to expand the cells. Techniques well known to those skilled in the art can be found in R. I. Freshney, Ed., ANIMAL CELL CULTURE: A PRACTICAL APPROACH (IRL Press, Oxford England, 1986) and R. I. Freshney, Ed., CULTURE OF ANIMAL CELLS: A MANUAL OF BASIC TECHNIQUES, Alan R. Liss & Co., New York, 1987).

The medium can be any medium suited for the growth of primary fibroblast cultures. In most instances, the medium is supplement with serum in the amount of between 0.5% and 20% (v/v) to promote growth of the fibroblasts. Higher concentrations of serum promote faster growth of the fibroblasts. In one variation, the serum is fetal bovine serum, which is added to a final concentration of 10% of medium. For example, the medium may include high glucose, DMEM supplemented with 2 mM glutamine, 110 mg/L sodium pyruvate, 10% (v/v) fetal bovine serum, and antibiotics (“complete medium”).

The cells can be passaged into new flasks by trypsinization. For expansion, individual flasks can be split 1:3. Triple bottom, T-150 flasks, having a total culture area of 450 cm² are suitable to use. A triple bottom T-150 can be seeded with about 6×10⁶ cells and has a capacity to yield about 1.8×10⁷ cells. When the capacity of the flask is reached, which typically requires 5-7 days of culture, the growth medium is replaced by serum-free complete medium; thereafter the cells are incubated, i.e., held at between about 30° C. and about 40° C., for at least 6 hours, preferably for greater than 12 hours and most preferably from 16-18 hours at 37° C., in the protein-free medium. The incubation of the cells in serum free medium substantially removes from the cells the proteins that are derived from the fetal bovine serum which, if present, would be immunogenic in the subject and cause an allergic reaction.

After expansion, the cells are removed from the tissue culture flask by trypsin/EDTA; washed extensively by centrifugation and resuspension; and suspended in a carrier material, e.g., PRP or isotonic saline. The cells would not be suspended in a fibrin clot or cultured in a fibrin clot. Six triple bottom T-150 flasks, grown to capacity, yield about 10⁸ cells, which is sufficient to make up about 1.0 ml of suspension.

In some variations, the cells can be transported at 4° C., so long as they are used within 18 hours of the time that the suspension was made. The cells can be suspended in an equal volume of complete medium, except for the absence of phenol red pH indicator, and the replacement of the fetal bovine serum by the subject's serum for such transportation (transport medium). The cells can be aspirated and injected in the transport medium. The volume of saline or transport medium in which the cells are suspended is not critical and may depend upon such factors as the number of fibroblasts the physician desires to use, the size and number of the defects that are to be treated, and the urgency of the subject's desire to obtain the results of treatment.

Alternatively, instead of using a flask-based cell expansion system as described above, an autologous dermal fibroblast composition, as well as other autologous cell compositions (e.g., adipose derived mesenchymal cell compositions), can be prepared using an automated, closed cell expansion system such as the Quantum® Cell Expansion System (Terumo BCT, Inc., Lakewood, Colo.). Exemplary automated, closed cell expansion systems may employ hollow fiber cell growth bioreactors. These three dimensional bioreactors may comprise over 10,000 individual hollow fibers of about 200 microns in length, the inner surface of which may yield a total surface growth area of over two square meters (a typical large tissue culture flask may have between about 150 to about 175 square centimeters of growth area). It is understood that the total surface growth area may be adjusted by varying the number and/or length of the hollow fiber bioreactors employed. The large surface area of the automated, closed cell expansion systems are generally designed to maximize adherent cell (e.g., bone marrow cells, adipose cells, mesenchymal stem cells, amniocytes, epithelial cells, etc.) expansion in a minimal volume. Furthermore, the defined and stable geometry of the hollow fiber bioreactor may optimize control and management of the cell culture environment. The closed environment of the bioreactor generally remains intact throughout the culture process, eliminating open operation and hands-on tasks associated with traditional, flask-based tissue culture. Thus, such automated, closed cell expansion systems generally reduce the risk of operator error and contamination.

In other variations, a hybrid approach may be taken. For example, the primary fibroblast cultures may be established in a medium containing a small amount of fetal bovine serum (e.g., 2% to 3% fetal bovine serum) such as FGM™-2 Fibroblast Growth Medium (Lonza Group Ltd., Basel, Switzerland) for one or two passages in conventional T-Flasks and then expanded using an automated, closed system such as the Quantum® Cell Expansion System using serum-free media (e.g., a serum-free, chemically defined medium such as FGM-CD™ (Lonza Group Ltd., Basel, Switzerland).

### Extracelular Matrix Materials

In some variations of the methods described herein, the PRP composition and autologous cell composition may be supplemented with a biocompatible extracellular matrix (ECM) material. The ECM material can serve as a scaffold to retain or support the autologous cells at their site of administration. For example, the ECM material may retain or support the fibroblasts that are cultured and expanded as described above. In addition, the ECM material may facilitate migration of endogenous cells into the area of administration. The ECM material may be injected or topically applied to an area affected by a skin condition, followed by the administration of PRP and autologous cells. Alternatively, the PRP composition or autologous cell composition may be combined with the ECM material and then administered, e.g., by topical application or by injection.

The ECM material may comprise a natural, synthetic, or semisynthetic material, so long as it is biocompatible. Exemplary ECM materials include human amnion extracellular matrix and porcine urinary bladder matrix. Other ECM materials may include polymers. Suitable polymers may include without limitation, gelatin, hyaluronic acid, chitosan, polyglycolic acid, polylactic acid, polypropylenefumarate, polyethylene glycol, and copolymers and combinations thereof.

### Administration

The methods described herein may be used to cosmetically improve skin conditions, and generally employ the step of micro-needling prior to the administration of PRP and autologous cell compositions. However, the PRP with or without an autologous cell composition can also be used as a lubricant for the micro-needling device. As previously stated, micro-needling of an affected area improves absorption of the compositions into the area, and enhances the regeneration of tissue. In general, the methods for cosmetically improving skin conditions comprise the steps of: 1) obtaining a blood sample from a subject affected by a skin condition; 2) preparing a platelet-rich plasma composition from the blood sample; 3) preparing an autologous cell composition; and 4) performing a micro-needling procedure over an area of skin affected by the skin condition followed by topical application of the platelet-rich composition and the autologous cell composition to the affected area. In some variations, the autologous cell composition comprises fibroblasts. In other variations, the autologous cell composition comprises adipose cells. In yet further variations, the autologous cell composition comprises stem cells, e.g., adipose derived mesenchymal cells. In some instances, it may be useful to include fibroblasts and adipose derived mesenchymal cells in the autologous cell composition.

The micro-needling and administration of PRP and autologous cell compositions may be repeated every 4 to 6 weeks for 4 to 6 times, however other micro-needling and administration regimes may be implemented depending on the skin condition being treated and severity of the condition. Repeated micro-needling and PRP and autologous cell application may generally stimulate the cells previously administered. In some instances, it may be beneficial to apply heat via ultrasonic, radiofrequency, or laser devices to tissue where the PRP and cells have been previously applied. For example, the heat could be applied at a later date to cells that were administered previously (at a different office visit). The skin conditions that may be improved by such methods include without limitation, acne scars, traumatic scars, hyperpigmentation, fine lines, wrinkles, stretch marks, and hair loss.

When the PRP composition is used as a carrier, cultured autologous cells can be suspended in the PRP and then topically applied to a desired area, e.g., an area affected by a skin condition. In some variations, it may be beneficial to topically apply a PRP composition in which cultured fibroblasts have been suspended. In another variation, it may be beneficial to topically apply a PRP composition in which cultured adipose cells have been suspended. Alternatively, it may be beneficial to topically apply a PRP composition in which cultured adipose derived mesenchymal cells have been suspended. An extracellular matrix material that provides a scaffold for the autologous cells can further be added to the PRP composition or autologous cell composition, or separately applied to the affected area.

When not used as a carrier, the PRP composition and autologous cell composition can be layered in any desired order. For example, the platelet-rich plasma could be applied first followed by the autologous cell composition or vice versa. The layering of compositions can also be repeated as desired. In some variations, a layer of adipose cells or adipose derived mesenchymal cells is applied to the skin prior to micro-needling. In other variations, the adipose cells or adipose derived mesenchymal cells are combined with fibroblasts and then layered onto the skin prior to micro-needling. As noted above, enhanced tissue regeneration and improved skin appearance may be obtained by the application of PRP and one or more cell compositions.

A tissue sealant may be applied over any layer to help retain the topically applied PRP and/or autologous cell composition within the skin and thus improve their absorption. For example, TISSEEL® Fibrin Sealant (Baxter International Inc., Deerfield, Ill.) may be topically applied over one or more layers. In other instances, the PRP, autologous cell compositions, and/or extracellular matrix material are combined and injected into or beneath the skin, e.g., into the epidermis, dermis, or subcutaneous tissue to improve a skin condition.

The PRP compositions, autologous cell compositions, tissue sealants, cellular growth factors, and extracellular matrix materials may be administered to any area of the body where cosmetic improvement is desired. For example, the compositions and materials may be administered to areas of the face, neck, hands, etc. Administration will generally include topical application by spreading the compositions onto the skin, but could also include spray application.

In some instances, the compositions described herein are used as fillers, and are injected into the dermis or subcutaneous tissue. Here the volume of the compositions may help to improve cosmetic appearance or treat a skin condition. Injection of one or more compositions can be performed in addition to micro-needling and/or the layering of PRP and/or the layering of an autologous cell composition. Exemplary compositions for injection may include PRP, an autologous cell composition, conditioned media, ECM material comprising, e.g., human amnion or porcine urinary bladder, cellular growth factors, or combinations thereof. The autologous cell compositions may comprise any one or more of the cell types described herein, e.g., fibroblasts, adipose cells, and mesenchymal cells. In some variations, the injection of PRP and fibroblasts, PRP and adipose cells, or PRP and adipose derived mesenchymal cells may be useful. Other variations of the method may include the injection of conditioned media and fibroblasts, conditioned media and adipose cells, or conditioned media and adipose derived mesenchymal cells.

Injection may be beneficial when treating wrinkles or deep acne scars. In one specific variation where the skin condition is fine superficial facial lines, the affected area is prepped with alcohol and stretched to give a taut surface. A syringe is then filled with fibroblasts suspended in a PRP composition and fitted with a 30-gauge needle for injection. The needle is inserted into the skin site as superficially as possible; the orientation of the bevel is not critical. An intradermal injection is made by gentle pressure until a slight blanch is seen. Multiple serial injections are then made. A similar procedure may be performed for hair rejuvenation.

In further variations, the method for improving cosmetic appearance or improving skin conditions involves a multi-modality approach. Here the approach may consider a subject's need for tissue volume, need for lifting and toning of deep and superficial tissues, restoration of the dermis, and/or restoration of the epidermis. When tissue volume is needed, the approach may include the use of injectable fillers as described herein, cellular therapy (e.g., layering with one or more of the autologous cell compositions described herein), and/or the application of cellular growth factors. When improvement of skin laxity, skin tone, and/or dull complexion is desired, the approach may include any one or more of the following: micro-needling with an automated device as described herein, cellular therapy (e.g., layering with one or more of the autologous cell compositions described herein), application of cellular growth factors, radiofrequency micro-needling, deep ultrasound heating, and fractionated laser. Similarly, the appearance of fine lines and nasolabial folds may be improved by the use of injectable fillers as described herein, micro-needling with an automated device as described herein, cellular therapy (e.g., layering with one or more of the autologous cell compositions described herein), application of cellular growth factors, radiofrequency micro-needling, and/or deep ultrasound heating. The approach for improving stretch marks may include cellular therapy (e.g., layering with one or more of the autologous cell compositions described herein), micro-needling with an automated device as described herein, the application of cellular growth factors, and/or radiofrequency micro-needling. In some variations, hyperpigmentation can be improved using an automated micro-needling device as described herein and the application of cellular growth factors. When improvement of acne scars is desired, the approach may include any one or more of the following: fat transplants with their mesenchymal stem cells, subscision, cellular therapy (e.g., layering with one or more of the autologous cell compositions described herein), micro-needling with an automated device as described herein, the application of cellular growth factors, radiofrequency micro-needling, and laser resurfacing. When hair loss is to be improved, the approach may include micro-needling with an automated device as described herein, cellular therapy (e.g., layering with one or more of the autologous cell compositions described herein), and the application of cellular growth factors.

1. A method for cosmetically improving skin conditions, comprising:
obtaining a blood sample from a subject affected by a skin condition;
preparing a platelet-rich plasma composition from the blood sample;
preparing an autologous cell composition;
performing a micro-needling procedure over an area of skin affected by the skin condition followed by topical application of the platelet-rich composition and the autologous cell composition to the affected area.

2. The method of claim 1, wherein the micro-needling procedure is performed by an automated micro-needling device, the automated micro-needling device comprising a plurality of needles fixed on a disposable needle portion, and a reusable handle removably secured to the disposable needle portion.

3. The method of claim 2, wherein the automated micro-needling device further comprises a needle depth adjustment mechanism.

4. The method of claim 3, wherein performing the micro-needling procedure includes adjusting the depth of the plurality of needles using the depth adjustment mechanism.

5. The method of claim 4, wherein the depth of the plurality of needles is adjusted to between 0.5 mm and 2.75 mm.

6. The method of claim 1, wherein the platelet-rich plasma composition and autologous cell composition are mixed together and topically applied to the affected area.

7. The method of claim 1, wherein the platelet-rich plasma composition is applied to the affected area followed by application of the autologous cell composition.

8. The method of claim 7, further comprising repeat topical application of the platelet-rich plasma composition.

9. The method of claim 1, wherein the autologous cell composition is applied to the affected area followed by topical application of the platelet-rich plasma composition.

10. The method of claim 1, further comprising topically applying a tissue sealant over the platelet-rich plasma composition or the autologous cell composition.

11. The method of claim 1, wherein the autologous cell composition comprises adipose cells, adipose derived mesenchymal cells, chondrocytes, fibroblasts, keratinocytes, stem cells, or combinations thereof.

12. The method of claim 1, wherein the autologous cell composition comprises fibroblasts.

13. The method of claim 1, wherein the autologous cell composition comprises adipose cells.

14. The method of claim 1, wherein the autologous cell composition comprises adipose derived mesenchymal cells.

15. The method of claim 1, wherein the autologous cell composition comprises fibroblasts, and adipose cells or adipose derived mesenchymal cells.

16. The method of claim 1, further comprising applying a biocompatible extracellular matrix material to the affected area.

17. The method of claim 16, wherein the biocompatible extracellular matrix material comprises human amnion tissue matrix.

18. The method of claim 16, wherein the biocompatible extracellular matrix material comprises porcine urinary bladder matrix.

19. The method of claim 1, further comprising applying a lubricant to the area of skin prior to the micro-needling procedure.

20. The method of claim 19, wherein the lubricant comprises platelet-rich plasma, adipose cells, adipose derived mesenchymal cells, or combinations thereof.

21. The method of claim 1, wherein the skin conditions are selected from the group comprising acne scars, traumatic scars, hyperpigmentation, fine lines, wrinkles, stretch marks, and hair growth.

22. The method of claim 1, further comprising injecting an autologous cell composition into the affected area.

23. The method of claim 22, wherein the autologous cell composition comprises adipose cells, adipose derived mesenchymal cells, chondrocytes, fibroblasts, keratinocytes, stem cells, or combinations thereof.

24. The method of claim 22, wherein the autologous cell composition comprises fibroblasts.

25. The method of claim 22, wherein the autologous cell composition comprises adipose cells.

26. The method of claim 22, wherein the autologous cell composition comprises adipose derived mesenchymal cells.
